(19) United States
(12) Patent Application Publication
(10) Pub. No.: US 2009/0221800 A1
(43) Pub. Date: Sep. 3, 2009

(54) Method for the Production of Recombinant Proteins

(75) Inventors: Jan Nehlin, Greve (DK); Ivan Svendsen, Smørum (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk HealthCare A/G, Zurich (CH)

(21) Appl. No.: 12/464,973

(22) Filed: May 13, 2009

Related U.S. Application Data
(63) Continuation of application No. 11/481,169, filed on Jul. 5, 2006.
Application No. 11/481,169 is a continuation of application No. PCT/EP05/50060, filed on Jan. 7, 2005.
(60) Provisional application No. 60/536,376, filed on Jan. 14, 2004.

(30) Foreign Application Priority Data
Jan. 7, 2004 (DK) PA 2004 00015

(51) Int. Cl. C07K 14/745; C12P 21/02; C12N 5/10

(52) U.S. Cl. 530/384; 435/69.6; 435/372

(57) ABSTRACT

The present invention relates to a process for the production of recombinant polypeptides in leukocytes.

### CROSS-REFERENCE TO RELATED APPLICATIONS

This application is a continuation of U.S. application Ser. No. 11/481,169, filed Jul. 5, 2006 (pending), which is a continuation of International Application PCT/EP2005/050060, filed Jan. 7, 2005, which claimed priority of Danish Patent Application PA 2004 00015, filed Jan. 7, 2004, which further claimed priority under 35 U.S.C. § 119 of U.S. Provisional Application 60/536,376, filed Jan. 14, 2004.

### FIELD OF THE INVENTION

The present invention relates to methods for the production of recombinant proteins.

### BACKGROUND OF THE INVENTION

The blood coagulation cascade consists of a series of enzymatic reactions leading to the conversion of soluble plasma fibrinogen to fibrin clot. The coagulation factors are primarily synthesized in the liver and are either enzyme precursors (FXII, FXI, FX, thrombin) or cofactors (FV and FVIII). Coagulation is initiated by binding of activated factor VII (FVIIa) in plasma to tissue factor (TF), a glycoprotein which is expressed on the surface of cells in response to injury. The main role in vivo of the TF:FVIIa complex is to activate FIX, which together with FVIII can activate FX. Activated FX (FXa) amplifies the generation of thrombin that induces the formation of fibrin. Generally, the blood components which participate in what has been referred to as the coagulation “cascade” are proenzymes or zymogens, enzymatically inactive proteins which are converted to proteolytic enzymes by the action of an activator, itself an activated clotting factor. Coagulation factors that have undergone such a conversion are generally referred to as “active factors,” and are designated by the addition of a lower case “a” suffix (e.g., activated factor VII (FVIIa)).

Because of the many disadvantages of using human plasma as a source of pharmaceutical products, it is preferred to produce these proteins in recombinant systems. The clotting proteins, however, are subject to a variety of co- and post-translational modifications, including, e.g., asparagine-linked (N-linked) glycosylation; O-linked glycosylation; and γ-carboxylation of glu residues. For this reason, it is preferable to produce them in higher eukaryotic cells, which are able to modify the recombinant proteins appropriately.

FVII is normally synthesized in the liver but it has lately been found to be expressed in a number of other cell types and tissues including smooth muscle cells, macrophages, fibroblasts, keratinocytes and atherosclerotic plaques. Several industrial cell lines are capable of expressing recombinant human Factor VII in the absence of serum, such as BHK and CHO-K1 cells. Also, FVII transgenes have been efficiently expressed in mouse skeletal muscle cells following gene transfer. Factor VII has also been expressed from retroviral constructs expressed in mouse cells.

There is still a need in the art for alternative sources of human FVII production. The present invention provides this alternative source of FVII production, where FVII is expressed from leukocyte cells.

### SUMMARY OF THE INVENTION

The present invention relates in a broad aspect to the expression of recombinant FVII polypeptides in cells from the lymphoid lineage especially those cells that differentiate into B-cells or cancerous derivatives thereof, e.g. CLL (chronic lymphocytic leukemia), ALL (Acute lymphoblastic leukemia), CML (chronic myeloid leukemia), pre B-cell leukemia, Burkitts lymphoma, Multiple myeloma.

In a first aspect the present invention relates to a method for the production of a purified Factor VII polypeptide the method comprising:

(i) transfecting a leukocyte cell with a vector comprising a promoter sequence and a polynucleotide sequence coding for the Factor VII polypeptide;

(ii) cultivating the transformed host cell expressing the Factor VII polypeptide in a culture medium under conditions appropriate for expression of the Factor VII polypeptide;

(iii) recovering all or part of the culture medium comprising the Factor VII polypeptide; and

(iiii) purifying the Factor VII polypeptide from the culture medium.

In a second aspect the present invention relates to a leukocyte cell transformed with a vector comprising a promoter sequence and a polynucleotide sequence encoding a Factor VII polypeptide.

In a third aspect the present invention relates to a purified Factor VII polypeptide obtained by a method comprising:

(i) transfecting a leukocyte cell with a vector comprising a promoter sequence and a polynucleotide sequence coding for the Factor VII polypeptide;

(ii) cultivating the transformed host cell expressing the Factor VII polypeptide in a culture medium under conditions appropriate for expression of the Factor VII polypeptide;

(iii) recovering all or part of the culture medium comprising the Factor VII polypeptide; and

(iiii) purifying the Factor VII polypeptide from the culture medium.

In one embodiment of the invention, the leukocyte cell is a lymphoid cell.

### DESCRIPTION OF FIGURES

FIG. 1. Protein blots of FVII-secreted samples run under non-reducing conditions (FIG. 1A) or under reducing conditions (FIG. 1B) on 12% NuPage Bis-Tris polyacrylamide gels (Invitrogen Corp.). Lane 1 is a molecular size marker (Magic marker). Lane 2 is a sample derived from a control producer cell line, FVII-expressing hamster CHO-K1. Lanes 3-11 are samples derived from different FVII-expressing SP/0 myeloma cells selected with 800 micrograms/ml G418. Lane 13 is a sample derived from a FVII-expressing X63 myeloma cell line selected with 600 microgram/ml G418. Lanes 14 and 15 represent negative control samples derived from pcDNA3.1-transfected SP2/0 myeloma cells.

FIG. 2. Protein blot of FVII-Fc secreted samples run under non-reducing conditions (FIG. 2A) or under reducing conditions (FIG. 2B) on 12% NuPage Bis-Tris polyacrylamide gels (Invitrogen Corp.). Lane 1 is a molecular size marker (magic marker). Lane 2 is a FVII-Fc (FVII analogue)-expressing myeloma cell line.

FIG. 3. Protein blot of FVII-secreted samples treated with N-glycosidase F (PNGase). Panel A. Non-reducing conditions. Panel B. reducing conditions. Lanes 2-7 represent supernatants from diverse FVII-expressing cells, untreated (2,4,6) or treated with PNGase (3,5,7). Lane 1 is a molecular size marker (Magic marker). Lanes 2 and 3 derive from FVII-expressing myeloma SP2/0 cells. Lanes 3 and 4 derive from FVII-expressing hamster CHO-K1 producer cell line. Lanes 5 and 6 derive from a FVII-mutated expressing hamster CHO-K1 cell line devoid of glycans. The samples were all run on 12% NuPage Bis-Tris polyacrylamide gels (Invitrogen Corp.).

### DETAILED DESCRIPTION OF THE INVENTION

Factor VII (FVII) is a key protein that initiates the blood coagulation cascade. The formation of a complex between active FVII and tissue factor (TF) in response to injury triggers the formation of a blood clot. Expression of human recombinant FVII in established industrial cell lines is necessary for the production of FVII for therapeutic use.

The present invention describes the expression and production of various FVII polypeptides, examplified by the expression of human recombinant wild type FVII and a FVII fusion protein (FVII-Fc) in myeloma cells. High-producing clones were isolated that expressed active wild type human FVII and FVII-Fc fusion protein, both in serum-containing media and in media without serum, in the presence of vitamin K. Further increases in FVII polypeptide production may be obtained with cell fusions between the myeloma cell lines expressing FVII and FVII analogues and several other high-protein producing industrial cell lines.

Production of FVII for therapeutic use has been obtained in baby hamster kidney (BHK) cells and Chinese hamster ovary (CHO) cells cultured with or without the presence of serum. Serum-free production of FVII or other recombinant proteins often leads to productivity and cell viability losses, resulting in high costs and inefficient production rates.

The inventors of the present invention have found that myeloma cells are highly applicable for the production of FVII polypeptides. Myeloma cells expressing FVII polypeptides showed to be robust cell lines that can withstand growth in media without serum, allowing production of high levels of recombinant FVII without losses of cell viability.

The term “purified Factor VII polypeptide” as used herein, means a Factor VII polypeptide that has been separated from at least about 50 percent by weight of polynucleotides, lipids, carbohydrates and any other contaminating polypeptides or other contaminants that are found in the culture medium following expression in a eukaryotic host cells which would interfere with its therapeutic, diagnostic, prophylactic or research use. In one embodiment, the purified Factor VII polypeptide has been separated from at least about 60 such as 80, such as 90, such as 95, such as 99 percent by weight of polynucleotides, lipids, carbohydrates and any other contaminating polypeptides or other contaminants that are found in the culture medium following expression in a eukaryotic host cells. The Factor VII polypeptide can be purified to be substantially free of natural contaminants from the culture medium through the use of any of a variety of methodologies. Standard chromatographic separation technology for the purification of the Factor VII polypeptide may also be used in some of the purification steps.

By “purifying” a polypeptide from a composition comprising the polypeptide and one or more contaminants is meant increasing the degree of purity of the polypeptide in the composition by removing (completely or partially) at least one contaminant from the composition. A “purification step” may be part of an overall purification process resulting in a “homogeneous” composition, which is used herein to refer to a composition comprising at least about 70% by weight of the polypeptide of interest, based on total weight of the composition, preferably at least about 80% by weight.

The term “leukocyte cell” as used herein, means any cell existing or derived from nucleated cells that occur in blood or tissue fluid, exclusive of erythrocytes and erythrocyte precursors. The term includes hybridomas of leucocytes as well as the major clases of leukocytes including lymphoid cells such as B-, T- and NK (Natural killer) cells, monocytes including macrophages, and neutrophils, eosinophils and basophils. The term further includes myeloma cells, lymphoma cells, leukaemia cells and lymphoma cells. The term also includes the parental cells, including but not restricted to hematopoeitic stem cells (HSC) giving rise to the hematopoeitic cell lineages as described in Wagers and Weissman, 2004 Cell 116,639-648.

The term “leukemia cell” as used herein means any cell derived from a malignant leukocyte or derivatives thereof, any cell from the parental lineage leading to leukocyte formation, including but not restricted to any of several nucleated cells that naturally occur in blood or tissue fluid, such as lymphocytes, monocytes, granulocytes hereunder neutrophils, eosinophils, basophils and precursors of these cells.

The term “lymphoid cell” as used herein, means any cell derived from the lymphoid lineage. The term comprises all parental cells, and derivatives and hybridomas thereof, either primary or established cell lines, derived from human, non-human, non-primate species, including but not restricted to avian, amphibian, mammalian, reptile species, and/or derived from non-vertebrate species, including but not restricted to marine/aquatic organisms, insects, plants, lichens, moss, fungi. The term includes stem cells, differentiated cells, virus-transformed cells, cancerous cells, lymphoma cells, myeloma cells, leukemia cells and cell hybrids, any cell whose origin could be related with the lymphoid lineage, either in vertebrate or invertebrate species. The term includes lymphoid stem cells that give rise to the pre-B and pre-T cell lineages, the pre-B cells that give rise to B cells and thereafter actively Ig-producing plasma cells, as well as the pre-T cells that give rise to T cells and thereafter T helper, T suppressor and NK cells. Included within the term is any cancerous derivatives thereof, e.g. CLL (chronic lymphocytic leukemia), ALL (acute lymphoblastic leukemia), CML (chronic myeloid leukemia), pre B-cell leukemia, Burkitts lymphoma, Multiple myeloma.

The term “lymphoma cell” as used herein means any cell derived from a malignant neoplasm primarily affecting lymph nodes.

The term “myeloma cell” as used herein, means any malignant cell of bone marrow origin, including but not restricted to cells of B-lymphocyte lineage, such as CLL (chronic lymphocytic leukemia), ALL (Acute lymphoblastic leukemia), CML (chronic myeloid leukemia), pre B-cell leukemia, Burkitts lymphoma, Multiple myeloma, primary tumor cells, cells from established myeloma cell lines, hybrid cells produced from myeloma cells that retain the characteristic growth properties of myeloma cells, multiple myeloma, plasma cell myeloma, peripheral plasmacytoma, solitary plasmacytoma, and plasmoma.

The myeloma cell-line may be a rat, mouse, human or any other mammalian species myeloma or hybridoma cell-line, such as the rat YB2/3.0 Ag20 hybridoma cell-line, the mouse NS/O, NS-1 myeloma cell-lines or the mouse SP2/0-Ag14 hybridoma cell-line, MOPC-31C, P3X63Ag8.653, P3XAg8U.1, MPC-11, FO, Fox-NY, NS1, Human: RPMI 8226, IM-9, HS-Sultan, SKO-007, MC/CAR, HuNS1, NCI-H929, Human/Mouse: SHM-D33, A6, 36, mouse J558L myeloma cells, etc.

Rat hybridoma cell-line YB2/3.0 Ag20 is described in British patent specification 2079313 and is on deposit at the American Type Culture Collection (as YB2/O or YB2/3HL. P2. G11. 16Ag.20) under Accession Number CRL1662. Mouse hybridoma cell-line SP2-OAg14 is on deposit at the American Culture Collection under Accession Number CRL1581. Mouse hybridoma cell-line P3/NS1/1 Ag4.0 (the NS-1 cell-line) is on deposit at the American Culture Collection under Accession Number T1B18. Mouse myeloma P3X63Ag8.653 cell line is on deposit at the American Culture Collection under Accession Number CRL1580.

In one embodiment of the invention, the myeloma cell is selected from the group consisting of YB2/3.0 Ag20, SP2-OAg14, P3/NS1/1 Ag4.0, P3X63Ag8.653, mouse J558L myeloma cells, and mouse NS/O, NS-1 myeloma cell-lines.

Examples of other suitable cells include but are not limited to hormone-secreting cells, whether normal or tumorigenic, derived from blood, body fluids and tissues including but not restricted to pancreas, prostate gland, mammary gland, pituitary gland, hypothalamus, kidney, endocrine and exocrine glands, skin, muscle, vessels, either of human, primate, cow, pig, goal, sheep origin, and other vertebrate species.

Thus, in one further aspect, the invention relates to a transgenic animal containing a transformed cell of the invention. In one embodiment, the transformed cell is a mammary gland epithelial cell. In a further aspect, the invention relates to a method for producing the Factor VII polypeptide, the method comprising recovering the Factor VII polypeptide from milk produced by the transgenic animal.

Included are epithelial cells of mammary gland origin and their derivatives such as MCF10A (ATCC number CRL-10317), L612 (ATTC number CRL-10724), MCF-12A (ATCC number CRL-10782), MCF-7 (ATCC number HTB-22), BT-20 (ATCC number HTB-19), BT-474 (ATCC number HTB-20), MDA-MB-231 (ATCC number HTB-26), MDA-MB-436, SK-BR-3 (ATCC number HTB-30), MDA-MB-361 (ATCC number HTB-27), MDA-MB-157 (ATCC number HTB-24), MDA-MB-175-VII (ATCC number HTB-25), T-47D (ATCC number HTB-133), MDA-MB-468 (ATCC number HTB-132), MDA-MB-453 (ATCC number HTB-131), BT-549 (ATCC number HTB-122), DU4475 (ATCC number HTB-123), ZR-75-1 (ATCC number CRL-1500), cells from breast ductal carcinomas such as UACC-812 (ATCC number CRL-1897) and UACC-893 (ATCC number CRL-1902), HCC38 (ATCC number CRL-2314) and all other HCC cell lines, all other adenocarcinomas, ductal carcinomas, breast fibromas, and epithelial cells of mammary gland origin.

The list comprises all wild-type epithelial cells of mammary gland origin that either are capable of secreting b-casein and/or lactoferrin in their differentiated state, or epithelial cells of mammary gland origin that no longer express markers of differentiation typical of a mammary epithelial cell but are dedifferentiated, and could be defined as actively dividing, with increased expression of Id-1 (Singh et al. Oncogene, 2002, 21(12):1812-1822) and/or with mutations in BRCA1, with positive expression of estrogen and progesterone receptors. The list includes all cytokeratin 19 positive cells.

The list comprises also stem cells giving rise to the breast epithelial cell lineage including all cells expressing sca-1 (Stem-cell-antigen 1).

In one embodiment the method of the invention is a method, wherein the promoter is selected from the list consisting of cytomegalovirus promoter, metallothionein promoter, and adenovirus major late promoter.

In a further embodiment the method of the invention is a method, wherein the lymphoid cell is selected from the group consisting of CLL (chronic lymphocytic leukemia) cells, ALL (Acute lymphoblastic leukemia) cells, CML (chronic myeloid leukemia), pre B-cell leukemia cells, Burkitts lymphoma cells, Multiple myeloma cells, mouse myeloma cells, rat myeloma cells, human myeloma cells, fusion cell lines and transgenic myeloma cell lines.

In a further embodiment the method of the invention is a method, wherein the lymphoid cell is selected from the group consisting of YB2/3.0 Ag20, SP2-OAg14, P3/NS1/1 Ag4.0, P3X63Ag8.653, mouse J558L myeloma cells, and mouse NS/O, NS-1 hybridoma cell-lines, and transgenic myeloma cell lines with increased copy number of genes encoding proteins required for elevated protein expression, including mutated myeloma cell lines with enhanced productivity.

In a further embodiment the method of the invention is a method, wherein the lymphoid cell is selected from the group consisting of mouse myeloma cells, rat myeloma cells and human myeloma cells.

In a further embodiment the method of the invention is a method, wherein the lymphoid cell is selected from the group consisting of YB2/3.0 Ag20, SP2-OAg14, P3/NS1/1 Ag4.0, P3X63Ag8.653, mouse J558L myeloma cells, and mouse NS/O, NS-1 hybridoma cell-lines.

In a further embodiment the method of the invention is a method, wherein the Factor VII polypeptide is a compound having the formula A-(LM)-C, wherein A is a FVIIa polypeptide; LM is an optional linker moiety; C comprises an immunostimulatory effector domain; and wherein the compound binds to TF, as described in International patent application DK03/00481, which is hereby incorporated by reference in its entirety.

In a further embodiment the method of the invention is a method, wherein the transformed host cell expressing the Factor VII polypeptide is cultivated in a culture medium under conditions appropriate for expression of the Factor VII polypeptide and in the absence of serum. In one embodiment the cell cultures are cultivated in a medium lacking any animal derived components.

The methods of the present invention are particularly useful for large-scale production processes. By the term “large-scale” is typically meant methods wherein the volume of the liquid Factor VII polypeptide compositions is at least 100 L, such as at least 500 L, e.g. at least 1000 L, or at least 5000 L.

In a further embodiment of the invention, the Factor VII polypeptide is wild-type human factor VII.

In a further embodiment of the invention, the Factor VII polypeptide has a proteolytic activity higher than wild type human FVIIa.

In a further embodiment of the invention, the Factor VII polypeptide is selected from the group consisting of: L305V-FVII, L305V/M306D/D309S-FVII, L305T-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158 D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158 D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/L158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158 D/M298Q-FVII, F374Y/L305V/V158 D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158 D/E296V/M298Q/K337A/S314E-FVII, F374Y/L305V/V158 D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158 D/E296V/M298Q/K337A-FVII, F374Y/L305V/V158 D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys.

As used herein, “Factor VII polypeptide” encompasses wild-type Factor VII (i.e., a polypeptide having the amino acid sequence disclosed in U.S. Pat. No. 4,784,950), as well as variants of Factor VII exhibiting substantially the same or improved biological activity relative to wild-type Factor VII, Factor VII-related polypeptides as well as Factor VII derivatives, Factor VII conjugates, and FVII fusion proteins. The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. Such variants of Factor VII may exhibit different properties relative to human Factor VII, including stability, phospholipid binding, altered specific activity, and the like.

As used herein, “Factor VII-related polypeptides” encompasses polypeptides, including variants, in which the Factor VIIa biological activity has been substantially modified or reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The term includes conjugates of chemically inactivated wt-FVIIa with Fc domain as described in International patent application DK03/00481, which is incorporated by reference in its entirety. The term also includes dimers of FVII polypeptides, including variants, wherein the dimer is catalytically inactive as disclosed in International patent application 03/076461, which is incorporated by reference in its entirety.

The term “Factor VII derivative” as used herein, is intended to designate wild-type Factor VII, variants of Factor VII exhibiting substantially the same or improved biological activity relative to wild-type Factor VII and Factor VII-related polypeptides, in which one or more of the amino acids of the parent peptide have been chemically modified, e.g. by alkylation, PEGylation, acylation, ester formation or amide formation or the like. This includes but are not limited to PEGylated human Factor VIIa, cysteine-PEGylated human Factor VIIa and variants thereof.

The term “FVII fusion proteins” as used herein, means a FVII polypeptide, which is conjugated to another functional polypeptide. One example of such FVII fusion protein is a FVII-Fc, wherein the FVII polypeptide part of the protein is conjugated to the Fc portion of an antibody.

The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

The biological activity of Factor VIIa in blood clotting derives from its ability to (i) bind to tissue factor (TF) and (ii) catalyze the proteolytic cleavage of Factor IX or Factor X to produce activated Factor IX or X (Factor IXa or Xa, respectively). For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa to produce Factor Xa in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997) and (iv) measuring hydrolysis of a synthetic substrate.

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay, proteolysis assay, or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay, proteolysis assay, or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids.

Non-limiting examples of Factor VII variants having substantially the same or increased proteolytic activity compared to recombinant wild type human Factor VIIa include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189 (corresponding to WO 02/077218); and FVII variants exhibiting increased proteolytic stability as disclosed in WO 02/38162 (Scripps Research Institute); FVII variants having a modified GIa-domain and exhibiting an enhanced membrane binding as disclosed in WO 99/20767, U.S. Pat. No. 6,017,882 and U.S. Pat. No. 6,747,003, US Patent application 20030100506 (University of Minnesota) and WO 00/66753, US patent applications 20010018414, US 2004220106, and US 200131005, U.S. Pat. No. 6,762,286 and U.S. Pat. No. 6,693,075 (University of Minnesota); and FVII variants as disclosed in WO 01/58935, U.S. Pat. No. 6,806,063, US patent application 20030096338 (Maxygen ApS), WO 03/93465 (Maxygen ApS), WO 04/029091 (Maxygen ApS), WO 04/083361 (Maxygen ApS), and WO 04/111242 (Maxygen ApS), as well as in WO 04/108763 (Canadian Blood Services).

Non-limiting examples of FVII variants having increased biological activity compared to wild-type FVIIa include FVII variants as disclosed in WO 01/83725, WO 02/22776, WO 02/077218, PCT/DK02/00635 (corresponding to WO 03/027147), Danish patent application PA 2002 01423 (corresponding to WO 04/029090), Danish patent application PA 2001 01627 (corresponding to WO 03/027147); WO 02/38162 (Scripps Research Institute); and FVIIa variants with enhanced activity as disclosed in JP 2001061479 (Chemo-Sero-Therapeutic Res Inst.).

Examples of variants of factor VII include, without limitation, L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V1158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158 D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158 D/E296V-FVII, F374Y/L305V/V158 D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158 D/M298Q/S314E-FVII, F374Y/L305V/V158 D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158 D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn; FVII having substitutions, additions or deletions in the amino acid sequence from 304Arg to 329Cys; and FVII having substitutions, additions or deletions in the amino acid sequence from 153IIe to 223Arg.

The terminology for amino acid substitutions used are as follows. The first letter represents the amino acid naturally present at a position of human wild type FVII. The following number represents the position in human wild type FVII. The second letter represent the different amino acid substituting for (replacing) the natural amino acid. An example is M298Q, where a methionine at position 298 of human wild type FVII is replaced by a glutamine. In another example, V158T/M298Q, the valine in position 158 of human wild type FVII is replaced by a threonine and the methionine in position 298 of human wild type FVII is replaced by a Glutamine in the same Factor VII polypeptide.

In a further embodiment of the invention, the factor VII polypeptide is a polypeptide, wherein the ratio between the activity of the Factor VII polypeptide and the activity of the wild type human Factor VIIa is at least about 1.25. In one embodiment the ratio between the activity of the Factor VII polypeptide and the activity of the wild type human Factor VIIa is at least about 2.0. In a further embodiment the ratio between the activity of the Factor VII polypeptide and the activity of the wild type human Factor VIIa is at least about 4.0.

In a further embodiment of the invention, the factor VII polypeptide is a polypeptide, wherein the ratio between the activity of the Factor VII polypeptide and the activity of the wild type human Factor VIIa is at least about 1.25 when tested in a Factor VIIa activity assay. In one embodiment the ratio between the activity of the Factor VII polypeptide and the activity of the wild type human Factor VIIa is at least about 2.0 when tested in a Factor VIIa activity assay. In a further embodiment the ratio between the activity of the Factor VII polypeptide and the activity of the wild type human Factor VIIa is at least about 4.0 when tested in a Factor VIIa activity assay. The Factor VIIa activity may be measured by the assays described under “assays”.

In a further embodiment of the invention, the factor VII polypeptide is a polypeptide, wherein the ratio between the activity of the Factor VII polypeptide and the activity of the wild type human Factor VIIa is at least about 1.25 when tested in the “In Vitro Hydrolysis Assay”. In one embodiment the ratio between the activity of the Factor VII polypeptide and the activity of the wild type human Factor VIIa is at least about 2.0 when tested in the “In Vitro Hydrolysis Assay”. In a further embodiment the ratio between the activity of the Factor VII polypeptide and the activity of the wild type human Factor VIIa is at least about 4.0 when tested in the “In Vitro Hydrolysis Assay”.

In a further embodiment of the invention, the factor VII polypeptide is a polypeptide, wherein the ratio between the activity of the Factor VII polypeptide and the activity of the wild type human Factor VIIa is at least about 1.25 when tested in the “In Vitro Proteolysis Assay”. In one embodiment the ratio between the activity of the Factor VII polypeptide and the activity of the wild type human Factor VIIa is at least about 2.0 when tested in the “In Vitro Proteolysis Assay”. In a further embodiment the ratio between the activity of the Factor VII polypeptide and the activity of the wild type human Factor VIIa is at least about 4.0 when tested in the “In Vitro Proteolysis Assay”. In a further embodiment the ratio between the activity of the Factor VII polypeptide and the activity of the wild type human Factor VIIa is at least about 8.0 when tested in the “In Vitro Proteolysis Assay”.

The present invention is suitable for Factor VII/VIIa variants with increased activity compared to wild type. Factor VII/VIIa variants with increased activity may be found by testing in suitable assays described in the following. These assays can be performed as a simple preliminary in vitro test. Thus, the section “assays” discloses a simple test (entitled “In Vitro Hydrolysis Assay”) for the activity of Factor VIIa variants of the invention. Based thereon, Factor VIIa variants which are of particular interest are such variants where the ratio between the activity of the variant and the activity of wild type Factor VII is above 1.0, e.g. at least about 1.25, preferably at least about 2.0, such as at least about 3.0 or, even more preferred, at least about 4.0 when tested in the “In Vitro Hydrolysis Assay”.

The activity of the variants can also be measured using a physiological substrate such as factor X (“In Vitro Proteolysis Assay”) (see under “assays”), suitably at a concentration of 100-1000 nM, where the factor Xa generated is measured after the addition of a suitable chromogenic substrate (eg. S-2765). In addition, the activity assay may be run at physiological temperature.

The ability of the Factor VIIa variants to generate thrombin can also be measured in an assay comprising all relevant coagulation factors and inhibitors at physiological concentrations (minus factor VIII when mimicking hemophilia A conditions) and activated platelets (as described on p. 543 in Monroe et al. (1997) Brit. J. Haematol. 99, 542-547 which is hereby incorporated as reference).

The Factor VII polypeptides described herein are produced by means of recombinant nucleic acid techniques. In general, a cloned wild-type Factor VII nucleic acid sequence is modified to encode the desired protein. This modified sequence is then inserted into an expression vector, which is in turn transformed or transfected into host cells. The complete nucleotide and amino acid sequences for human Factor VII are known (see U.S. Pat. No. 4,784,950, where the cloning and expression of recombinant human Factor VII is described). The bovine Factor VII sequence is described in Takeya et al., J. Biol. Chem. 263:14868-14872 (1988)).

The amino acid sequence alterations may be accomplished by a variety of techniques. Modification of the nucleic acid sequence may be by site-specific mutagenesis. Techniques for site-specific mutagenesis are well known in the art and are described in, for example, Zoller and Smith (DNA 3:479-488, 1984) or “Splicing by extension overlap”, Horton et al., Gene 77, 1989, pp. 61-68. Thus, using the nucleotide and amino acid sequences of Factor VII, one may introduce the alteration(s) of choice. Likewise, procedures for preparing a DNA construct using polymerase chain reaction using specific primers are well known to persons skilled in the art (cf. PCR Protocols, 1990, Academic Press, San Diego, Calif., USA).

The nucleic acid construct encoding the Factor VII polypeptide of the invention may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., Molecular Cloning: A Laboratory Manual, 2nd. Ed. Cold Spring Harbor Laboratory, Cold Spring Harbor, N.Y., 1989).

The nucleic acid construct encoding the Factor VII polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesised, e.g. in an automatic DNA synthesiser, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct is preferably a DNA construct. DNA sequences for use in producing Factor VII polypeptides according to the present invention will typically encode a pre-pro polypeptide at the amino-terminus of Factor VII to obtain proper posttranslational processing (e.g. gamma-carboxylation of glutamic acid residues) and secretion from the host cell. The pre-pro polypeptide may be that of Factor VII or another vitamin K-dependent plasma protein, such as Factor IX, Factor X, prothrombin, protein C or protein S. As will be appreciated by those skilled in the art, additional modifications can be made in the amino acid sequence of the Factor VII polypeptides where those modifications do not significantly impair the ability of the protein to act as a coagulant. For example, the Factor VII polypeptides can also be modified in the activation cleavage site to inhibit the conversion of zymogen Factor VII into its activated two-chain form, as generally described in U.S. Pat. No. 5,288,629.

Expression vectors for use in expressing Factor VIIa variants will comprise a promoter capable of directing the transcription of a cloned gene or cDNA causing gene expression in animal cells (e.g., a SV40 promoter, a BPV promoter, a metallothionein promoter, a dhfr promoter, various long terminal repeat of retrovirus or LTRs all of which are well known). Preferred promoters include viral promoters and cellular promoters. Viral promoters include the SV40 promoter (Subramani et al., Mol. Cell. Biol. 1:854-864, 1981) and the CMV promoter (Boshart et al., Cell 41:521-530, 1985). A particularly preferred viral promoter is the major late promoter from adenovirus 2 (Kaufman and Sharp, Mol. Cell. Biol. 2:1304-1319, 1982). Cellular promoters include the mouse kappa gene promoter (Bergman et al., Proc. Natl. Acad. Sci. USA 81:7041-7045, 1983) and the mouse VH promoter (Loh et al., Cell 33:85-93, 1983). A particularly preferred cellular promoter is the mouse metallothionein-I promoter (Palmiter et al., Science 222:809-814, 1983). Expression vectors may also contain a set of RNA splice sites located downstream from the promoter and upstream from the insertion site for the Factor VII sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 Elb region, the human growth hormone gene terminator (DeNoto et al. Nucl. Acids Res. 9:3719-3730, 1981) or the polyadenylation signal from the human Factor VII gene or the bovine Factor VII gene. The expression vectors may also include a noncoding viral leader sequence, such as the adenovirus 2 tripartite leader, located between the promoter and the RNA splice sites; and enhancer sequences, such as the SV40 enhancer.

Cloned DNA sequences are introduced into cultured myeloma cells by, for example, calcium phosphate-mediated transfection (Wigler et al., Cell 14:725-732, 1978; Corsaro and Pearson, Somatic Cell Genetics 7:603-616, 1981; Graham and Van der Eb, Virology 52d:456-467, 1973), cationic liposome-mediated transfection (Felgner et al., Proc. Natl. Acad. Sci. 84:7413-7417), electroporation (Neumann et al., EMBO J. 1:841-845, 1982) or infection by retroviral or viral-based expression vectors.

High-level expression of Factor VII polypeptides of interest can also be achieved by means of an IRES element (Internal ribosome entry sequence) that functions as translation enhancer regions derived from 5′-non coding areas of varios genes (e.g. use thereof explained in U.S. Pat. No. 4,937,190).

For references on all transfection methods please see Sambrook and Russell (2001) Molecular cloning: a laboratory manual. Cold Spring Harbor Laboratory Press.

To identify and select cells that express the exogenous DNA, a gene that confers a selectable phenotype (a selectable marker) is generally introduced into cells along with the gene or cDNA of interest. Preferred selectable markers include genes that confer resistance to drugs such as neomycin, hygromycin, and methotrexate. The selectable marker may be an amplifiable selectable marker. A preferred amplifiable selectable marker is a dihydrofolate reductase (DHFR) sequence. Selectable markers are reviewed by Thilly (Mammalian Cell Technology, Butterworth Publishers, Stoneham, Mass., incorporated herein by reference). The person skilled in the art will easily be able to choose suitable selectable markers.

Selectable markers may be introduced into the cell on a separate plasmid at the same time as the gene of interest, or they may be introduced on the same plasmid. If, on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells. After the cells have taken up the DNA, they are grown in an appropriate growth medium, typically for 1-2 days, to begin expressing the gene of interest. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991, Freshney, R. I. ed. Culture of animal cells, John Wiley & Sons, 2001, for mammalian cell culture protocols and media). Growth media generally include a carbon source, a nitrogen source, essential amino acids, essential sugars, vitamins, salts, phospholipids, proteins and growth factors. For production of gamma-carboxylated Factor VII polypeptides, the medium will contain vitamin K, preferably at a concentration of about 0.1 mg/ml to about 5 mg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the desired Factor VII polypeptide.

Transgenic animal technology may be employed to produce the Factor VII polypeptides of the invention. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and biochemically well characterized. Furthermore, the major milk proteins are present in milk at high concentrations (typically from about 1 to 15 g/l).

From a commercial point of view, it is clearly preferable to use as the host a species that has a large milk yield. While smaller animals such as mice and rats can be used (and are preferred at the proof of principle stage), it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk (see, for example, WO 88/00239 for a comparison of factors influencing the choice of host species). It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489), beta lactoglobulin, a lactalbumin, and whey acidic protein. The beta lactoglobulin (BLG) promoter is preferred. In the case of the ovine beta lactoglobulin gene, a region of at least the proximal 406 bp of 5′ flanking sequence of the gene will generally be used, although larger portions of the 5′ flanking sequence, up to about 5 kbp, are preferred, such as a ˜4.25 kbp DNA segment encompassing the 5′ flanking promoter and non coding portion of the beta lactoglobulin gene (see Whitelaw et al., Biochem. J. 286: 31 39 (1992)). Similar fragments of promoter DNA from other species are also suitable.

Other regions of the beta lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836 840 (1988); Palmiter et al., Proc. Natl. Acad. Sci. USA 88: 478 482 (1991); Whitelaw et al., Transgenic Res. 1: 3 13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta lactoglobulin gene, is preferred. One such region is a DNA segment that provides for intron splicing and RNA polyadenylation from the 3′ non coding region of the ovine beta lactoglobulin gene. When substituted for the natural 3′ non coding sequences of a gene, this ovine beta lactoglobulin segment can both enhance and stabilize expression levels of the protein or polypeptide of interest. Within other embodiments, the region surrounding the initiation ATG of the variant Factor VII sequence is replaced with corresponding sequences from a milk specific protein gene. Such replacement provides a putative tissue specific initiation environment to enhance expression. It is convenient to replace the entire variant Factor VII pre pro and 5′ non coding sequences with those of, for example, the BLG gene, although smaller regions may be replaced.

For expression of Factor VII polypeptides in transgenic animals, a DNA segment encoding variant Factor VII is operably linked to additional DNA segments required for its expression to produce expression units. Such additional segments include the above mentioned promoter, as well as sequences that provide for termination of transcription and polyadenylation of mRNA. The expression units will further include a DNA segment encoding a secretory signal sequence operably linked to the segment encoding modified Factor VII. The secretory signal sequence may be a native Factor VII secretory signal sequence or may be that of another protein, such as a milk protein (see, for example, von Heijne, Nucl. Acids Res. 14: 4683 4690 (1986); and Meade et al., U.S. Pat. No. 4,873,316, which are incorporated herein by reference).

Construction of expression units for use in transgenic animals is conveniently carried out by inserting a variant Factor VII sequence into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of a variant Factor VII polypeptide; thereby creating a gene fusion that includes the expression control sequences of the milk protein gene. In any event, cloning of the expression units in plasmids or other vectors facilitates the amplification of the variant Factor VII sequence. Amplification is conveniently carried out in bacterial (e.g. E. coli) host cells, thus the vectors will typically include an origin of replication and a selectable marker functional in bacterial host cells. The expression unit is then introduced into fertilized eggs (including early stage embryos) of the chosen host species. Introduction of heterologous DNA can be accomplished by one of several routes, including microinjection (e.g. U.S. Pat. No. 4,873,191), retroviral infection (Jaenisch, Science 240: 1468 1474 (1988)) or site directed integration using embryonic stem (ES) cells (reviewed by Bradley et al., Bio/Technology 10: 534 539 (1992)). The eggs are then implanted into the oviducts or uteri of pseudopregnant females and allowed to develop to term. Offspring carrying the introduced DNA in their germ line can pass the DNA on to their progeny in the normal, Mendelian fashion, allowing the development of transgenic herds. General procedures for producing transgenic animals are known in the art (see, for example, Hogan et al., Manipulating the Mouse Embryo: A Laboratory Manual, Cold Spring Harbor Laboratory, 1986; Simons et al., Bio/Technology 6: 179 183 (1988); Wall et al., Biol. Reprod. 32: 645 651 (1985); Buhler et al., Bio/Technology 8: 140 143 (1990); Ebert et al., Bio/Technology 9: 835 838 (1991); Krimpenfort et al., Bio/Technology 9: 844 847 (1991); Wall et al., J. Cell. Biochem. 49: 113 120 (1992); U.S. Pat. No. 4,873,191; U.S. Pat. No. 4,873,316; WO 88/00239, WO 90/05188, WO 92/11757; and GB 87/00458). Techniques for introducing foreign DNA sequences into mammals and their germ cells were originally developed in the mouse (see, e.g., Gordon et al., Proc. Natl. Acad. Sci. USA 77: 7380 7384 (1980); Gordon and Ruddle, Science 214: 1244 1246 (1981); Palmiter and Brinster, Cell 41: 343 345 (1985); Brinster et al., Proc. Natl. Acad. Sci. USA 82: 4438 4442 (1985); and Hogan et al. (ibid.)). These techniques were subsequently adapted for use with larger animals, including livestock species (see, e.g., WO 88/00239, WO 90/05188, and WO 92/11757; and Simons et al., Bio/Technology 6: 179 183 (1988)). To summarise, in the most efficient route used to date in the generation of transgenic mice or livestock, several hundred linear molecules of the DNA of interest are injected into one of the pro nuclei of a fertilized egg according to established techniques. Injection of DNA into the cytoplasm of a zygote can also be employed.

The Factor VII polypeptides of the invention are recovered from cell culture medium or milk. The Factor VII polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). Preferably, they may be purified by affinity chromatography on an anti-Factor VII antibody column. The use of calcium-dependent monoclonal antibodies is described by Wakabayashi et al., J. Biol. Chem. 261:11097-11108, (1986) and Thim et al., Biochemistry 27: 7785-7793, (1988). Additional purification may be achieved by conventional chemical purification means, such as high performance liquid chromatography. Other methods of purification, including barium citrate precipitation, are known in the art, and may be applied to the purification of the novel Factor VII polypeptides described herein (see, for example, Scopes, R., Protein Purification, Springer-Verlag, N.Y., 1982).

For therapeutic purposes it is preferred that the Factor VII polypeptides of the invention are substantially pure. Thus, in a preferred embodiment of the invention the Factor VII polypeptides of the invention is purified to at least about 90 to 95% homogeneity, preferably to at least about 98% homogeneity. Purity may be assessed by e.g. gel electrophoresis and amino-terminal amino acid sequencing.

The Factor VII polypeptide is cleaved at its activation site in order to convert it to its two-chain form. Activation may be carried out according to procedures known in the art, such as those disclosed by Osterud, et al., Biochemistry 11:2853-2857 (1972); Thomas, U.S. Pat. No. 4,456,591; Hedner and Kisiel, J. Clin. Invest. 71:1836-1841 (1983); or Kisiel and Fujikawa, Behring Inst. Mitt. 73:29-42 (1983). Alternatively, as described by Bjoern et al. (Research Disclosure, 269 September 1986, pp. 564-565), Factor VII may be activated by passing it through an ion-exchange chromatography column, such as Mono Q (Pharmacia fine Chemicals) or the like. The resulting activated Factor VII polypeptide may then be formulated and administered as described below.

### Assays

### In Vitro Hydrolysis Assay

Wild type (native) Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). The chromogenic substrate D-IIe-Pro-Arg-p-nitroanilide (S-2288, Chromogenix, Sweden), final concentration 1 mM, is added to Factor VIIa (final concentration 100 nM) in 50 mM Hepes, pH 7.4, containing 0.1 M NaCl, 5 mM CaCl₂ and 1 mg/ml bovine serum albumin. The absorbance at 405 nm is measured continuously in a SpectraMax® 340 plate reader (Molecular Devices, USA). The absorbance developed during a 20-minute incubation, after subtraction of the absorbance in a blank well containing no enzyme, is used to calculate the ratio between the activities of variant and wild-type Factor VIIa:

Ratio=(A405 nm Factor VIIa variant)/(A405 nm Factor VIIa wild-type).

In Vitro Proteolysis Assay

Wild type (native) Factor VIIa and Factor VIIa variant (both hereafter referred to as “Factor VIIa”) are assayed in parallel to directly compare their specific activities. The assay is carried out in a microtiter plate (MaxiSorp, Nunc, Denmark). Factor VIIa (10 nM) and Factor X (0.8 microM) in 100 microL 50 mM Hepes, pH 7.4, containing 0.1 M NaCl, 5 mM CaCl₂ and 1 mg/ml bovine serum albumin, are incubated for 15 min. Factor X cleavage is then stopped by the addition of 50 microL 50 mM Hepes, pH 7.4, containing 0.1 M NaCl, 20 mM EDTA and 1 mg/ml bovine serum albumin. The amount of Factor Xa generated is measured by addition of the chromogenic substrate Z-D-Arg-Gly-Arg-p-nitroanilide (S-2765, Chromogenix, Sweden), final concentration 0.5 mM. The absorbance at 405 nm is measured continuously in a SpectraMax® 340 plate reader (Molecular Devices, USA). The absorbance developed during 10 minutes, after subtraction of the absorbance in a blank well containing no FVIIa, is used to calculate the ratio between the proteolytic activities of variant and wild-type Factor VIIa:

Ratio=(A405 nm Factor VIIa variant)/(A405 nm Factor VIIa wild-type).

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The present invention is further described by the following examples which should not be construed as limiting the scope of the invention.

### EXAMPLES

We describe hereafter the procedures followed to show that FVII and its related analogues can be successfully expressed in cells that normally do not express FVII, that is, in cells whose origin and function is not involved in the FVII coagulation cascade. Moreover, it is shown that FVII and its analogues can be expressed abundantly and in their active form in a manner that is comparable to existing producer cell lines.

The practice of the present invention is based on conventional techniques that are within the skill of the art. Unless otherwise indicated, the methods in molecular biology necessary to generate plasmids, hereunder recombinant DNA cloning and microbiology techniques, are described in detail in Sambrook & Russell, Molecular Cloning, A Laboratory manual (2001); Ausubel et al. (eds), Short protocols in molecular biology (2002), etc. Cell biology-related techniques are widely described in Cellis, Cell biology: a laboratory handbook (1997); Freshney, Culture of animal cells: a manual of basic technique 4th ed. (2000); Hardin et al. Cloning, gene expression and protein purification: experimental procedures and process rationale (2001), and related sources.

In the following examples it is to be understood, that the process may be used for any FVII polypeptide according to the present invention.

### Example 1

### Expression of Human FVII and FVII Analogues in Myeloma Cells

Plasmids were first purified using the Qiagen Maxi Prep plasmid purification kits. Transfection of plasmids containing cDNAs encoding human FVII (pTS8) and FVII analogues (eg. pTS72) into myeloma cell lines P3X63Ag8.653 and SP2-OAg14 (also referred to as X63 and SP2/0) was performed.

Two myeloma cell lines, SP2/0 and X63 were stably transfected using Lipofectamine 2000 (Invitrogen Corp) with purified plasmids encoding human FVII and FVII-Fc (a FVII analogue)(see above), as well as control plasmids pcDNA3.1 (Invitrogen Corp,) and pIRESneo-2b (Stratagene). The selection of stable transfectants was done at 800 micrograms/ml G418 (Geneticin, Cat. Nr. 10131-019 Gibco/invitrogen).

### Example 2

### Clonal Selection of High-FVII and FVII Analogues-Producing Cell Lines

Limited dilution of stable transfectants was performed in 96-well plates, reaching a concentration of 1-10 cells/well. The highest producing cell populations (>300 ng/ml FVII) were chosen. Clones from myeloma cell lines SP2/0 and X63 have the ability to express both recombinant human FVII and the fusion protein FVII-Fc, as judged from ELISA-based assays and Western blotting. Relevant cell pools (from the 96-well plates) were expanded to 6-well plates, confirmed as positive by ELISA, and expanded further to 25 cc flasks and afterwards to 175 cc flasks. Secreted FVII amounts was measured in the order of >1 mg/ml.

### Example 3

### Determination of Activity of FVII and FVII-Analogues Secreted by Transgenic Myeloma Cell Lines

Supernatants from various FVII expressing myeloma cell lines were obtained directly and analyzed by Western blotting. The cell supernatants were loaded onto Nupage 12% Bis-Tris acrylamide gels, separated by electrophoresis, transferred to PVDF membranes (Invitrogen Corp.) and incubated with several FVII-specific polyclonal antibodies raised against various parts of the FVII molecule. FVII expressed in myeloma cells is glycosylated, as well as FVII expressed in CHO-K1 cells. Treatment of myeloma FVII-expressing cells with N-glycosidase F (PNGase; New England Biolabs), which hydrolizes all types of N-glycan chain in glycopeptides, results in a FVII of lower molecular weight, devoid of carbohydrates. This finding suggests that myeloma cells do indeed express a post-translationally modified FVII, as is the case in CHO-K1 cells expressing FVII.

An activity assay was carried out on myeloma clones expressing plasmid controls or FVII and/or FVII analogues (FVII-Fc), in the absence or presence of vitamin K (5 microgram/ml) in the media. The assay is based on how efficient FVII can promote coagulation in plasma using thromboplastin as described in U.S. Pat. No. 5,997,864. Lack of vitamin K in the cell media resulted in non-detectable active FVII, whereas myeloma cells grown with vitamin K (5 microgram/ml), express active FVII and active FVII analogues (FVII-Fc). Sp2/0 cells express more active, higher levels of FVII, than X-63 cells. Cells expressing plasmid controls (pcDNA3.1 and pIRESneo-2b) do not have any detectable FVII activity, as expected. Myeloma cells expressing FVII and FVII analogues (FVII-Fc) were grown in media without serum, in the presence of vitamin K, and initial measurements suggest that myeloma cells can efficiently express active FVII and FVII analogues (FVII-Fc).

### Example 4

### Fusion of Myeloma Cells with Themselves or with Other Cell Types

Myeloma cells expressing FVII and/or FVII analogues (FVII-Fc) were electrofused (cell fusion chamber/multiporator Eppendorf) with themselves or to liver cells and/or other organ specific cells, and stable polyploid cell clones were generated. The resulting clones were screened for their ability to express active human FVII and FVII analogues (FVII-Fc), in the presence of vitamin K. The fusion of two independent cell lines resulted in new cell lines with improved capacity to express FVII and FVII analogues (FVII-Fc), as judged from ELISA-based assays, Western blotting and clotting assays. Myeloma cells may be fused either by classical fusion methods or by electrofusion (Langonem J. J. and van Vunakis, H. editors, Immunochemical techniques, Methods in Enzymology, Volume 121, Academic Press, 1986; Bartal, A. H. and Hirshaut, Y. editors, hybridoma formation: methods and mechanisms, Humana Press, 1987).

### Example 5

### Expression of Human FVII and FVII Analogues in Myeloma Cells

Transfection of plasmids containing cDNAs encoding human FVII (pTS8) and human FVII analogues (eg. pTS72) as well as control plasmids pcDNA3.1 (Invitrogen Corp,) and pIRESneo-2b (Stratagene) into myeloma cell lines P3X63Ag8.653 and SP2-OAg14 was performed. It is shown that biologically active FVII and FVII analogues can be successfully expressed in myeloma cells.

### Selection of Expressing Clones

Prior to transfection of expression vectors carrying cDNA's encoding for FVII or FVII analogues, and selection of expressing cell clones using an antibiotic-resistance marker, the host cell lines needed to be checked for potential resistance to the antibiotic itself, without carrying any antibiotic-resistance gene or exogenously-derived cDNA. Two myeloma cell lines were chosen for the study, P3X63Ag8.653 and SP2-OAg14 (referred to hereafter as X63 and SP2/0). Myeloma cells were seeded out onto 96-well plates, cultured in DMEM media with Glutamax (Invitrogen), 10% fetal calf serum and containing a range of G418 (Geneticin; Invitrogen Corp.) concentrations of 100, 200, 300, 400, 500, 600, 700, 800, 900, and 1000 μg/ml. Their survival ability was followed during at least 10 days. Visual inspection of the surviving clones led us to conclude that a concentration of 600 μg/ml G418 is an appropriate amount that could kill all existing cells. Thus, any selection of exogenously introduced expression vectors carrying a G418 antibiotic marker would have to be done at a minimum of 600 μg/ml, for the chosen myeloma cells, to be able to eliminate any non-desired untransfected cells.

### Plasmid Preparation and Transfection

The following plasmids were first purified using the Maxi Prep plasmid purification kits (Qiagen): plasmid pTS8 containing a cDNA encoding human FVII, plasmid pTS72 containing a cDNA encoding FVII-Fc, a FVII analogue, control/parental plasmid pcDNA3.1 (Invitrogen Corp.) of pTS8 and control/parental plasmid pIRESneo-2b (Stratagene) of pTS72. The two myeloma cell lines, SP2/0 and X63 were stably transfected using Lipofectamine 2000 (Invitrogen Corp) according to the manufacturer's instructions with the above purified plasmids. Each transfection was performed in 5 ml Opti-Mem medium without fetal calf serum and antibiotics during 48 h in 6-well dishes with a cell density of 1.0×10⁶ c/ml. Opti-MEM is a modification of Eagle's minimum essential medium supplemented with hypoxanthine, thymidine, sodium pyruvate, L-Glutamine, trace elements and growth factors (Invitrogen Corp.). The transfection media was carefully discarded and new DMEM media with 10% fetal calf serum, Glutamax, 5 microgram/ml vitamin K and antibiotic G418 was added. The selection of stable transfectants was done at both 600 and 800 micrograms/ml G418 (Geneticin, Invitrogen Corp.). Media changes were performed once every Monday, Wednesday and Friday. A higher concentration of G418 (800 micrograms/ml) would result in a stronger selection of stable clones expressing FVII, while restricting the growth of clones whose plasmid copy number and resulting FVII expression is not high enough, as would be expected from the selection of clones in media containing 600 microgram/ml G418.

An initial FVII-quick test assay was performed on the transfectants and it was confirmed that FVII can be expressed in myeloma cells (Table 1).

TABLE 1
FVII-activity (FVII:C) in supernatant samples
from diverse FVII-expressing cell lines

| Sample | | FVII | Expected | Analysis |
|---|---|---|---|---|
| nr. | Sample | μg/ml | U/ml | U/ml |
| # 7 | SP2/0, pTS8 (FVII), with | 0.4 | 0.8 | 0.46 |
| | vitamin K | | | |
| # 15 | SP2/0, pTS8 (FVII), with | 0.4 | 0.8 | 0.33 |
| | vitamin K | | | |
| # 19 | SP2/0, pTS8 (FVII), with | 0.5 | 1.0 | 0.51 |
| | vitamin K | | | |
| # 7 | SP2/0, FVII, without | 0.4 | 0.8 | No |
| | vitamin K | | | measurable |
| | | | | activity |
| # 38 | SP2/0, pcDNA3-1, control, | — | — | No |
| | with vitamin K | | | measurable |
| | | | | activity |
| # 26 | X-63, pTS72 (FVII-Fc), with | 0.1 | 0.2 | 0.13 |
| | vitamin K | | | |
| — | X-63, pIRESneo, 2B, control, | — | — | No |
| | with vitamin K | | | measurable |
| | | | | activity |
| Normal | | 0.5 | 1.0-1.1 | 1.0 |
| human | | | | |
| plasma | | | | |

### Example 6

### Clonal Selection of High-FVII and FVII Analogues-Producing Cell Lines

Approximately 2 weeks after transfection, the transfected cells were subject to limited dilution, whereby cells are counted and diluted out in order to attempt getting approximately 1-10 cell clones/well in 96-well plates. pTS8 (FVII-expressing) plasmid-transfected SP2/0 cells were seeded out onto 4 96-well plates and selected with 600 and 800 micrograms/ml G418 (2 plates each). The same was done for pTS72 (FVII-Fc-expressing) transfected cells. pTS8 (FVII-expressing) plasmid-transfected X63 cells were seeded onto 8 96-well plates and selected with 600 and 800 micrograms/ml G418 (4 plates each). Control plates carrying single-transfected cell clones of the parental/control plasmids were also tested. Approximately 10 days after performing the limited dilution procedure, 100 microliters of cell supernatants from wells where growth could be detected were transferred to 96-well assay plates and subject to a FVII-quick Elisa test, to measure the concentration of secreted FVII. It was possible to detect FVII (up to 390 ng/ml) in a number of wells. Further incubation of the cell clones in the 96-well plates resulted, as expected, in much higher FVII concentrations (up to 1070 ng/ml) especially for SP2/0 cells selected with 800 microgram/ml G418. Cell clones expressing FVII amounts higher than 150 ng/ml were transferred to 6-well plates (100 microliters of cell suspension to 5 ml new media per well). The levels of secreted FVII were maintained during growth in 6-well plates. After 10-20 days of growth (approximately 2 months post-transfection), cells were transferred to 25 cc cell culture flasks. Measurement of FVII concentration in the supernatants showed high levels in many of the clones (up to 1270 ng/ml) indicating that the production of FVII remains relatively stable with levels correlated with cell density. The selection of stable transfectants was done at 800 micrograms/ml G418.

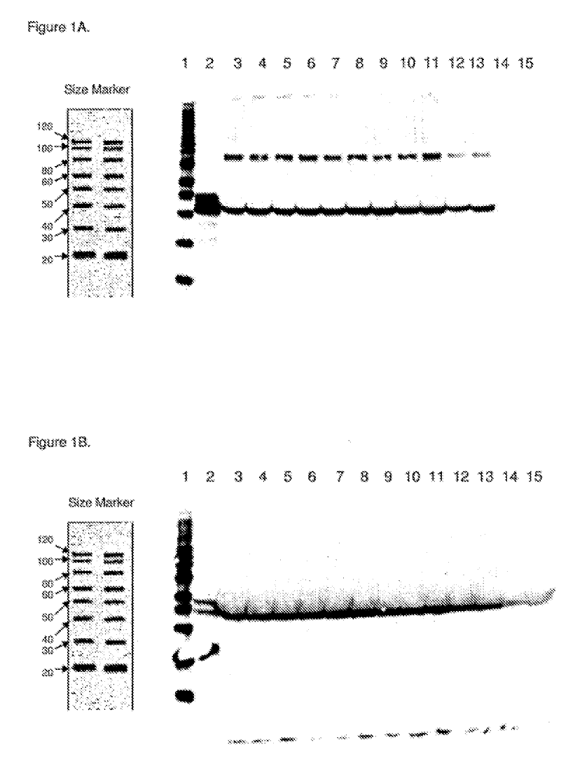

In summary, clones from myeloma cell lines SP2/0 and X63 have the ability to express both recombinant human FVII and the fusion protein FVII-Fc, as judged from ELISA-based assays and Western blotting (see FIGS. 1A, 1B and Table 2). Relevant cell pools (from the 96-well plates) were expanded to 6-well plates, confirmed as positive by ELISA, and expanded further to 25 cc flasks and afterwards to 175 cc flasks. Secreted FVII amounts were measured in the order of >1 mg/ml. It could also be concluded that FVII expression was higher in SP2/0 myeloma cells than in X63 myeloma cells as judged from FVII Elisa assays (data not shown).

### Protein Characterization

A Western blot procedure (protein blot) was run under reducing and non-reducing conditions to detect FVII. One single band was obtained that is similar to a positive control, a FVII-analogue expressed in CHO-K1 cells. However, the patterns are different under reducing conditions. This could be due to the fact that vitamin K was not added in the original media. However, vitamin K addition did not change the resulting profiles in a retest of the above mentioned Western under reducing or non-reducing conditions.

Myeloma cell pools from the 25 cc flasks showing the highest FVII levels, as judged by ELISA, were chosen and expanded to 75 cc flasks. A protein gel was run under reducing and non-reducing conditions to detect FVII by the Western blot procedure (FIGS. 1A and B). A single band of the same size as a positive control was obtained (FIGS. 1A,B).

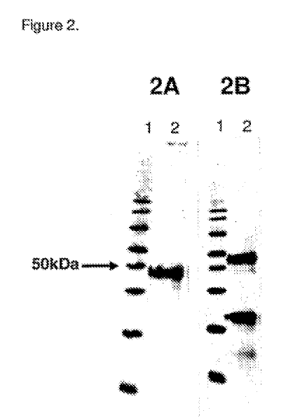

Myeloma cell pools expressing the FVII-Fc analogue were also harvested and protein extracts were subject to FVII-protein blots. A representative example is shown in FIG. 2A (non-reducing conditions) and in FIG. 2B (reducing conditions).

Protein samples derived from cell culture supernatants (20 microliters) were prepared either under non-reducing or reducing conditions, and denatured at 72 degrees C. during 10 min before loading onto NuPage 12% Bis-Tris acrylamide gels in a Novex XCell II MiniCell system (Invitrogen Corp.) and electrophoresed at 200 volts during 0.5-1 hour. A molecular size marker, Magic Marker, was used in the runs. The protein ladders were subsequently transferred to a nitrocellulose membrane using the Blot module of the Novex XCell II MiniCell system at 24-28 volts during 1.5 hours at room temperature. The nitrocellulose membrane was blocked with wash buffer containing 2% Tween 20 during 2 min and incubated with rabbit anti-human FVII polyclonal IgG primary antibody at a concentration of 0.2 microgram/ml, and a goat anti-mouse IgG-HRP conjugated secondary antibody at a 1:2000 dilution. Chemiluminescence was detected by using a Fuji luminescence scanner.

### Example 7

### Determination of Activity of FVII and FVII-Analogues Secreted by Transgenic Myeloma Cell Lines

It has been shown that myeloma cells have the ability to express FVII and FVII-derived analogue polypeptides judging from the analysis of protein blots, as well as from the ELISA analysis showing the presence of immunoreactive, FVII-specific signals. However, in order to demonstrate that the polypeptides being secreted by the transgenic myeloma cells are biologically active and functional, a series of assays were undertaken. Several myeloma clones expressing FVII and the FVII-Fc analogue were subject to a coagulation assay to detect biological activity and a glycosidation assay to monitor post-translational modifications.

### Coagulation Assay

An activity assay was carried out on myeloma clones expressing plasmid controls or FVII and/or FVII analogues (FVII-Fc), in the absence or presence of vitamin K (5 microgram/ml) in the media. The assay is based on how efficient FVII can promote coagulation in plasma using thromboplastin as described in U.S. Pat. No. 5,997,864. Lack of vitamin K in the cell media resulted in non-detectable active FVII, whereas myeloma cells grown with vitamin K (5 microgram/ml), express active FVII and active FVII analogues (FVII-Fc). Sp2/0 cells express more active, higher levels of FVII, than X-63 cells. Cells expressing plasmid controls (pcDNA3.1 and pIRESneo-2b) do not have any detectable FVII activity, as expected.

Supernatants from the 25 cc flasks were used to measure the clotting activity of the FVII being produced (Table 1). Normal human plasma containing 0.5 microgram/ml FVII is expected to give 1-1.1 U/ml FVII clotting activity. Myeloma SP2/0 cells expressing 0.5 microgram/ml of FVII showed a clotting activity of up to 0.51 U/ml (Table 1), indicating that myeloma cells are indeed capable of expressing functionally active FVII, although to a lesser degree than normal human plasma. The contribution of vitamin K to the activity of FVII has been reported previously (see reviews by Berkner, 2000, J. Nutr. 130:1877-1880; Suttie, 1992, J. Am. Diet Assoc. 92:585-590). Thus, measurement of clotting activity in myeloma SP2/0 cells expressing FVII in the absence of vitamin K in the culture media resulted in no measurable FVII activity (Table 1), indicating the essential role vitamin K plays as a cofactor in FVII expression. FVII expression was not detectable all along in those myeloma cells transfected with parental/control plasmids in media with or without vitamin K (Table 1). Coagulation activity of expressed FVII-Fc, a FVII analogue was lower than for FVII, (Table 1).

Myeloma cells expressing FVII and FVII analogues (FVII-Fc) were grown in media without serum, in the presence of vitamin K, and initial measurements suggest that myeloma cells can efficiently express active FVII and FVII analogues (FVII-Fc).

The one-step coagulation assay for measurement of FVII activity (FVII:C) in human plasma was performed according to standard operating procedures at Novo Nordisk, using an ACL300/3000 research instrument, as described in Broze & Majerus, Human Factor VII, Methods Enzymol. 80:228-237 (1981). Briefly, the test sample's ability to normalize coagulation time is measured in a one-step system consisting of Factor VII-deprived plasma (Helena Labs) and rabbit thromboplastin (Manchester reagent). Coagulation is started by addition of thromboplastin-Ca⁺⁺ reagent.

### Glycosidation Assay

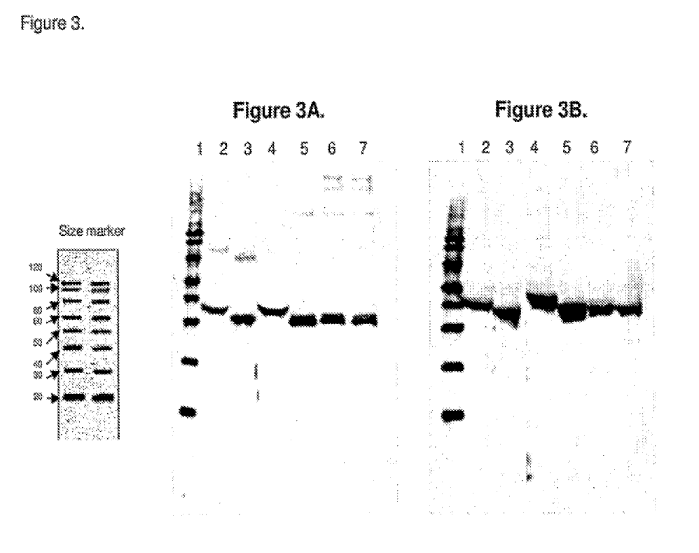

Supernatants from various FVII-expressing myeloma cell lines were obtained directly and analyzed by Western blotting (FIGS. 3A and B). The cell supernatants were loaded onto Nupage 12% Bis-Tris acrylamide gels, separated by electrophoresis, transferred to PVDF membranes (Invitrogen Corp.) and incubated with a rabbit anti-human FVII polyclonal antibody. FVII expressed in myeloma cells is glycosylated, as well as FVII expressed in CHO-K1 cells (lanes 2,4, FIGS. 3A and B). Treatment of myeloma FVII-expressing cells with N-glycosidase F (PNGase; New England Biolabs), which hydrolizes all types of N-glycan chain in glycopeptides, results in a FVII of lower molecular weight, devoid of glycans (lanes 3,5, FIGS. 3A and B). This finding suggests that myeloma cells do indeed express a post-translationally modified FVII (FIG. 3 A,B, lane 2), as is the case for CHO-K1 cells expressing FVII (FIG. 3 A,B, lane 4). A mutant FVII than cannot undergo glycation, exhibits the lower molecular weight band, even in the presence of PNGase (FIG. 3 A,B, lanes 6,7). These results further indicate that FVII can undergo normal protein processing in myeloma cells as judged from the ability of such cells to appropriately glycate recombinant FVII.

1. A method for the production of a purified Factor VII polypeptide, said method comprising:
(i) transforming a leukocyte cell with a vector comprising a promoter and a polynucleotide sequence coding for a Factor VII polypeptide;
(ii) cultivating the said leukocyte cell expressing said Factor VII polypeptide in a culture medium under conditions appropriate for expression of said Factor VII polypeptide;
(iii) recovering all or part of the culture medium comprising said Factor VII polypeptide; and
(iiii) purifying said Factor VII polypeptide from the culture medium,
wherein said Factor VII polypeptide is selected from the group consisting of wild-type human Factor VII and variants of wild-type human Factor VII exhibiting substantially the same or improved biological activity relative to wild-type Factor VII.

2. The method according to claim 1, wherein said leukocyte cell is a lymphoid cell.

3. The method according to claim 1, wherein said promoter is selected from the group consisting of cytomegalovirus promoter, metallothionein promoter, and adenovirus major late promoter.

4. The method according to claim 1, wherein said leukocyte cell is selected from the group consisting of CLL (chronic lymphocytic leukemia) cells, ALL (acute lymphoblastic leukemia) cells, CML (chronic myeloid leukemia), pre B-cell leukemia cells, Burkitts lymphoma cells, multiple myeloma cells, mouse myeloma cells, rat myeloma cells, human myeloma cells, fusion cell lines, YB2/3.0 Ag20, SP2-OAg14, P3/NS1/1 Ag4.0, P3X63Ag8.653, mouse NS/O, NS-1 hybridoma cell-lines, and transgenic myeloma cell lines.

5. The method according to claim 4, wherein said leukocyte cell is a transgenic myeloma cell line with increased copy number of genes encoding proteins required for elevated protein expression.

6. The method according to claim 1, wherein said Factor VII polypeptide is wild-type human factor VII.

7. The method according to claim 1, wherein said Factor VII polypeptide is a variant relative to wild-type human Factor VII selected from the group consisting of: L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V 158T-FVII, L305V/K337A/M298Q-FVII, L305V/K337A/E296V-FVII, L305V/K337A/V 158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V 158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V 158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V 158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V 158D-FVII, F374Y/V 158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V 158T/M298Q-FVII, F374Y/K337A/V 158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V 158D/E296V/M298Q-FVII, F374Y/L305V/V 158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V 158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V 158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; FVII having substitutions, additions or deletions in the amino acid sequence from 233Thr to 240Asn, and FVII having substitutions, additions or deletions in the amino acid sequence from 304Arg to 329Cys.

8. A leukocyte cell transformed with a vector comprising (i) a promoter and (ii) a polynucleotide sequence encoding a Factor VII polypeptide.

9. The leukocyte cell according to claim 8, wherein said leukocyte cell is a lymphoid cell.

10. The leukocyte cell according to claim 9, wherein said lymphoid cell is selected from the group consisting of CLL (chronic lymphocytic leukemia) cells, ALL (acute lymphoblastic leukemia) cells, CML (chronic myeloid leukemia), pre B-cell leukemia cells, Burkitts lymphoma cells, multiple myeloma cells, mouse myeloma cells, rat myeloma cells, human myeloma cells, fusion cell lines, YB2/3.0 Ag20, SP2-OAg14, P3/NS1/1 Ag4.0, P3X63Ag8.653, mouse NS/O, NS-1 hybridoma cell-lines, and transgenic myeloma cell lines.

11. The leukocyte cell according to claim 8, wherein said Factor VII polypeptide is wild-type human factor VII.

12. The leukocyte cell according to claim 8, wherein said Factor VII polypeptide is a variant relative to wild-type human Factor VII selected from the group consisting of: L305V-FVII, L305V/M306D/D309S-FVII, L305I-FVII, L305T-FVII, F374P-FVII, V158T/M298Q-FVII, V 158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V 158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V 158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V 158D-FVII, F374Y/K337A/V 158T/M298Q-FVII, F374Y/K337A/V 158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V 158D/E296V/S314E-FVII, F374Y/V 158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V 158T/E296V/K337A-FVII, F374Y/L305V/V 158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; FVII having substitutions, additions or deletions in the amino acid sequence from 233Thr to 240Asn, FVII having substitutions, additions or deletions in the amino acid sequence from 304Arg to 329Cys.

13. A purified Factor VII polypeptide obtained by a method according to claim 1.
